(19) United States
(12) Patent Application Publication
(10) Pub. No.: US 2022/0064627 A1
(43) Pub. Date: Mar. 3, 2022

(54) MICROFLUIDIC DEVICE

(71) Applicant: HAPLOMIC TECHNOLOGIES PTY LTD, Windsor Gardens (AU)

(72) Inventors: Matthew Daniel Solomon, Windsor Gardens (AU); Richard Walter Doumani, Windsor Gardens (AU)

(21) Appl. No.: 17/418,603

(22) PCT Filed: Jan. 23, 2020
(86) PCT No.: PCT/AU2020/050042

§ 371 Date: Jun. 25, 2021

(30) Foreign Application Priority Data
Jan. 23, 2019 (AU) 2019900210

(51) Int. Cl. C12N 15/10; B01L 3/00; C12Q 1/6806

(52) U.S. Cl. CPC C12N 15/1003; B01L 3/502746; C12Q 1/6806; B01L 2400/024; B01L 2300/14; B01L 2200/0652; B01L 2300/0877

(57) ABSTRACT

The present disclosure relates to a microfluidic device for the separation of metaphase chromosomes such that individual metaphase chromosomes may be dispensed discretely from the device. The microfluidic device comprises a flow channel including a series of expanded regions and constrictions. The present disclosure also relates to methods of separating metaphase chromosomes.

### CROSS REFERENCE TO RELATED APPLICATIONS

This application claims the benefit of Australian Patent Application No. 2019900210 filed 23 Jan. 2019, the entirety of which is incorporated herein by reference.

### FIELD OF THE INVENTION

The invention relates to a microfluidic device for separation of chromosomes.

### BACKGROUND OF THE INVENTION

The cells of many eukaryotes, including animal and plant species, contain more than one set of chromosomes, with the number of sets known as ploidy. For example, humans are diploid, possessing paired sets of chromosomes (maternal and paternal copies) that form the genome. At each position or locus for a specific chromosome, an individual may either have two copies of the same sequence (such as a gene allele, mutation, marker, or epigenetic component), or two different sequences, with one version on each of the pair of chromosomes. Determining whether sequence elements such as gene alleles, mutations, markers, or epigenetic components from different loci occur together on the same member of a chromosome pair (in cis arrangement) or on opposite members of a chromosome pair (in trans arrangement) is known as phasing. When two or more sequences (alleles, mutations, markers, or epigenetics) occur in cis, this is known as a haplotype. Variations in sequence of these haplotypes can result in functional differences, such as differences in gene expression, protein function, and disease. As such, knowing the phasing or haplotypes of an individual can lead to understanding and control of biological pathways, such as improved diagnostic methods and/or methods of treatment. Unfortunately, there are a number of shortcomings with existing processes for phasing and haplotype determination.

In a review article by Quake et al. (Nature Methods, Vol. 11, No. 1, 2014, pp 19-21), Quake identifies that although genome “analysis has progressed from determining the reference sequence for the ‘average’ human genome to prolific sequencing of personal genomes”, some aspects of genomic analysis remain difficult. In particular, Quake goes on to state that existing conventional techniques are not well suited to haplotype determination.

Dolez̆el al. (Funct. Integr. Genomics, 2012, 12:397-416) discusses a range of approaches for separating and isolating individual chromosomes. One approach discussed by Dolez̆el includes the separation of chromosomes based on relative density, such as via gradient centrifugation; however, this approach has a number of shortcomings as it provides only for the separation of small and large chromosomes and is not suited to the isolation of particular chromosomes. Another approach discussed in Dolez̆el is the use of magnetic beads that are functionalised with chromosome specific probes; however, a shortcoming of this approach is that the isolated fractions are of low purity. Dolez̆el goes on to state that the most successful approach is the use of flow cytometry. In flow cytometry, droplets of dye-stained chromosomes are ejected from a flow chamber and passed through a laser beam where the scattered light is analysed to identify chromosomes of interest in the chromosome containing droplets according to light scatter and fluorescence, and deflecting those droplets using an electric field into a collection container. However, Dolez̆el discusses that flow cytometry is unable to resolve all chromosomes in various animal species (including humans, dogs, swine, and chicken). Dolez̆el goes on to state that a number of research groups have focussed their efforts on improving flow cytometry for separating and isolating individual chromosomes.

Another technique is the approach adopted in Fan et al. (Nat. Biotechnol., January 2011, 29(1):51-57). Fan et al. identify current techniques (such as mate-pair shotgun genome sequencing, various forms of polymerase chain reaction (PCR), atomic force microscopy with carbon nanotubes, fosmid/cosmid cloning, and the use of hybridized probes) all have a number of significant shortcomings that prevent widespread adoption. Instead, Fan et al. report the development of a microfluidic device for separating and amplifying homologous copies of each chromosome from a single human metaphase cell. The device of Fan et al. is divided into five different regions according to their function. The first region includes the use of an optical microscope to identify a single metaphase cell. Once the metaphase cell has been identified, a series of surrounding valves are actuated to capture the cell so that the cell can be introduced into the second region of the device. In the second region the metaphase cell is contacted with pepsin to digest the cytoplasm of the cell and form a chromosome suspension. This suspension is then passed into the third region where it is partitioned by actuating a series of valves within the device into 48 chambers. In the fourth region, the contents of each of the 48 chambers are then individually amplified on device through a series of distinct channels via treatment with trypsin, alkali, and subsequent neutralisation for multiple strand displacement amplification. The fifth region of the device includes separate outlet ports for collection of each of the amplified chromosomes.

Notably, to the best knowledge of the inventors, the device disclosed in Fan et al. has not been adopted. The inventors have attempted to replicate protocols reported by Fan et al. with no success. The inventors speculate that the lack of repeatability of the device and process of Fan et al. has prevented adoption. In this regard, Dolez̆el suggests a departure from the microfluidic device of Fan et al. stating that the “application of flow cytogenetics may be an elegant alternative to the recently developed microfluidic approach, in which individual chromosomes from a single human metaphase are separated into distinct channels and amplified (Fan et al. 2011).”

Because of the shortcomings in technology to separate chromosomes for direct phasing, most current attempts to deduce phasing or haplotypes (for example, to match bone marrow transplant patients with prospective donors) use indirect methods of inference or assumption, such as family segregation studies, linkage disequilibrium or algorithms for producing probabilities of phasing from sequenced DNA fragments.

In the metaphase state, chromosomes constitute discrete bundles of tightly folded DNA and proteins. Such chromosomes may form associations with each other and be present in the form of a cluster of chromosomes.

In view of the above, there is a need for the development of a device and/or process for sorting and separating chromosomes to enable direct phasing and haplotype determination. However, there are significant shortcomings with the approaches of the prior art. It is thus an object of the invention to address and/or ameliorate one or more shortcomings of the prior art.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect of the invention there is provided a microfluidic device for separating metaphase chromosomes in a metaphase chromosome-containing fluid, the microfluidic device including:

a flow channel including:
an inlet to receive a fluid including metaphase chromosomes;
an outlet to discretely dispense individual metaphase chromosomes;
a series of expanded regions; and
one or more constrictions located between consecutive expanded regions
in the series of expanded regions;

wherein the constrictions are operable to apply sufficient shear stress to separate the metaphase chromosomes from one another; and

the expanded regions are operable to disperse chromosomes from one another.

In a further aspect of the invention there is provided a microfluidic device for separating clustered metaphase chromosomes within a fluid, the microfluidic device including:

a flow channel including:
an inlet to receive the fluid;
an outlet to dispense the separated metaphase chromosomes;
one or more expanded regions; and
one or more constrictions with at least one expanded region downstream of a constricted region;

wherein the constrictions are operable to apply sufficient shear stress to separate the clustered metaphase chromosomes; and

wherein the expanded regions are operable to disperse the separated metaphase chromosomes.

By ‘operable’ it is meant that the microfluidic device is operated under flow and/or pressure conditions such that the chromosomes are subjected to relatively high shear stress in the constrictions and/or relatively low flow velocity in the expanded regions (relative to the flow velocity in the non-expanded regions of the flow channel). For example, the device may be operated at a constant pressure where the change in flow velocity in the constrictions and expansions results in the respective shear stress and dispersal; or variable pressure such that when the chromosomes flow through the constrictions a pressure pulse is applied to subject the chromosomes to the shear stress, and when the chromosomes flow through the expanded regions the velocity decrease permits the chromosomes to disperse.

In one form of the invention, the metaphase chromosomes are in the form of one or more clusters of metaphase chromosomes, and the microfluidic device is for separating the one or more clusters of metaphase chromosomes into individual metaphase chromosomes. In such cases, the constrictions are operable to apply sufficient shear stress to the one or more clusters of metaphase chromosomes to separate metaphase chromosomes from the cluster or to break the cluster into smaller clusters; and the expanded regions are operable to disperse separated metaphase chromosomes and/or one or more clusters of metaphase chromosomes from one another.

By ‘clusters’ or ‘clustered’ it is meant a grouping or aggregation of metaphase chromosomes in which the metaphase chromosomes ‘stick’ or are closely associate with one another. This clustering may arise as the result of a number of physicochemical interactions, for example clustering can occur as chromosomes may form associations with each other, either directly (through protein or DNA interactions) or because of the presence of material such as cytoplasmic matrix. Therefore, chromosomes in fluid, particularly when associated with other cellular contents, may tend to stick or clump together.

In one form of the invention, the expanded regions are operable to disperse the separated metaphase chromosomes from one another.

As will be understood, a substantial proportion, or preferably all, of the metaphase chromosomes dispensed from the outlet of the device are discretely dispensed individual metaphase chromosomes.

In one form of the invention, the fluid including metaphase chromosomes is the lysate, or a component of the lysate, from a metaphase cell or cells, including in a fluid preparation.

As used herein, a ‘fluid’ may include dissolved materials. For instance, a fluid may include dissolved components of a buffer, such as a lysis buffer and/or a separation buffer.

In a further aspect of the invention, there is provided a microfluidic device for separating metaphase chromosomes in a metaphase chromosome-containing fluid, the microfluidic device including:

a flow channel having a width of from about 10 μm to about 30 μm, the flow channel including:

an inlet;

an outlet; and

a series of expanded regions, and one or more constrictions located between consecutive expanded regions in the series of expanded regions;

wherein the plurality of expanded regions have a channel width of from about 50 μm to about 150 μm, and each constriction in the plurality of constrictions has a minimum width of from about 1 μm to about 3 μm.

The sizing of the minimum widths of each constriction is to impede the passage of the chromosomes, requiring sufficient pressure to subject the chromosome to shear stress to drive the metaphase chromosomes through the constriction and to separate the metaphase chromosomes from one another.

The sizing of the expanded regions is to disperse separated metaphase chromosomes in both the transverse and axial directions via one or more of diffusion and advection which assists in increasing spacing between the chromosomes when they exit the expanded portion. The expanded regions may take any suitable size and shape.

In one form of the invention, the metaphase chromosomes are in the form of one or more clusters of chromosomes, and the microfluidic device is for separating the one or more clusters of metaphase into individual metaphase chromosomes.

In an embodiment of the above aspects of the invention, the flow channel (optionally other than the expanded regions and/or the constrictions and/or regions of the flow channel immediately adjacent the constrictions) has a depth of from about 5 μm up to about 40 μm. Preferably, the flow channel depth is from about 12 μm. More preferably, the flow channel depth is from about 14 μm. Even more preferably, the flow channel depth is from about 16 μm. Most preferably, the flow channel depth is from about 18 μm. Alternatively or additionally the flow channel depth is up to 35 μm. More preferably, the flow channel depth is up to about 30 μm. Most preferably, the flow channel depth is up to about 25 μm. In one non-limiting example, the flow channel depth is 20 μm±2 μm.

In an embodiment, the constrictions have a depth that is less than the depth of the flow channel. Preferably, the depth of the constrictions is from about 5 μm to about 15 μm less than the depth of the flow channel. It is preferred that the depth of the constrictions may be from about 5 μm to about 15 μm. The lesser depth of the constrictions relative to the flow channel is useful to increase the shear that the chromosomes are subjected to in the constrictions.

In one form of the above embodiment, there is a step change in depth between the bulk depth of the flow channel and the constriction depth. Preferably the step change in depth is from about 5 μm to about 15 μm, for example, a constriction depth of about 5 μm when the flow channel has a bulk depth of 10 μm or greater. It is also preferred that regions of the flow channel immediately adjacent to the constriction have the same depth as the constriction, such that the step change in depth is within the flow channel.

In one form of the above embodiment, the depth of the flow channel (other than the expanded regions and/or the constrictions and/or regions of the flow channel immediately adjacent the constrictions) is constant along a length of the flow channel. That is, the depth of the flow channel does not substantially vary along the length of the flow channel, such as to within ±2 μm.

In an embodiment of the above aspects of the invention, the flow channel (other than the expanded regions and the constrictions) has a width of from about 10 μm to about 30 μm. Preferably, the flow channel width is from about 12 μm. More preferably, the flow channel width is from about 14 μm. Most preferably, the flow channel width is from about 16 μm. Alternatively or additionally the flow channel width is up to 28 μm. More preferably, the flow channel width is up to about 26 μm. Most preferably, the flow channel width is up to about 24 μm. In one non-limiting example, the flow channel width is 20 μm±2 μm.

As will be understood, the geometry of the flow channel is selected as suitable for the particular application. For example, the width of the flow channel may be greater than the depth of the flow channel, or vice versa.

In an embodiment of the above aspects of the invention, the length of the flow channel is from about 2 mm to about 15 mm. Preferably, the flow channel length is from about 3 mm. Most preferably, the flow channel length is from about 4 mm. Alternatively or additionally the flow channel length is up to 12 mm. More preferably, the flow channel length is up to about 10 mm. Most preferably, the flow channel length is up to about 8 mm. In one non-limiting example, the flow channel length is approximately 5 mm.

In an embodiment, the minimum width of one or more constrictions, or of each constriction, is from about 1.00 μm to about 3.00 μm. Preferably, the minimum width is from about 1.25 μm. More preferably, the minimum width is from about 1.50 μm. Most preferably the minimum width is from about 1.75 μm. Alternatively or additionally the minimum width is up to about 2.75 μm. More preferably, the minimum width is up to about 2.50 μm. Most preferably, the minimum width is up to about 2.25 μm. In one non-limiting example, the minimum width is 2.00 μm±0.20 μm.

In an embodiment, the length of the minimum width portion of the constriction is from about 4 μm up to about 16 μm. Preferably, the length is from about 6 μm. Most preferably, the length is from about 8 μm. Alternatively, or additionally, it is preferred that the length is up to about 14 μm. Most preferably, up to about 12 μm. In one non-limiting example, the length is about 10 μm.

In an embodiment, each successive constriction from the inlet to the outlet has a smaller minimum width and/or depth than a preceding constriction. In an embodiment, at least some of the successive constrictions have the same minimum width and/or depth.

In an embodiment, one or more of the one or more constrictions has a widening tapered outlet. Preferably, the widening tapered outlet widens to a width that is about two-thirds the width of the flow channel or less. Preferably, the widening tapered outlet widens to a width that is half of the width of the flow channel or less.

In an embodiment, the expanded regions have a width that is from about 50 μm to about 150 μm. Preferably, the width of the expanded region is from about 60 μm. More preferably, the width of the expanded region is from about 70 μm. Most preferably, the width of the expanded region is from about 80 μm. Alternatively or additionally the width of the expanded region is up to 140 μm. More preferably, the width of the expanded region is up to about 130 μm. Most preferably, the width of the expanded region is up to about 120 μm. In one non-limiting example, the width of the expanded region is about 100 μm.

In an embodiment, the length of the expanded region is from about 0.2 mm up to about 0.8 mm. Preferably, the length is from about 0.3 mm. Most preferably, the length is from about 0.4 mm. Alternatively, or additionally, it is preferred that the length is up to about 0.7 mm. Most preferably, up to about 0.6 mm. In one non-limiting example, the length is about 5 mm.

In an embodiment, each of the expanded regions in the series of expanded regions has substantially the same width.

In an embodiment, the flow channel includes at least 3 expanded regions in the series of expanded regions. Preferably, at least 4 expanded regions. Most preferably, at least 5 expanded regions. Alternatively, or additionally, the flow channel includes up to 20 expanded regions. In preferred forms, the flow channel includes a number of expanded regions selected from: 5, 6, 7, 8, 9, 10, 11, 12, 13, 14, 15, 16, 17, 18, or 19.

In an embodiment, the flow channel includes one or more constrictions between each expanded region in the series of expanded regions.

In an embodiment, the inlet of the flow channel has a width of from 2 μm to 3 μm. Preferably, the constrictions have widths narrower than the inlet.

In an embodiment, the outlet of the flow channel has a width of from about 1 μm to 2 μm.

In an embodiment, the microfluidic device further includes cell capture and lysis structure upstream of the inlet, the cell capture and lysis structure including:

a cell trap adjacent the flow channel inlet configured to receive and retain a cell from a fluid sample including the cell, the cell trap including:
a viewing window to permit inspection of the cell; and
an opening connected to the flow channel inlet, the opening sized to impede passage of the cell therethrough;
a lysis port configured to introduce a lysis buffer to the cell trap.

In an embodiment, the size of the opening and the passage is from about 2 μm to about 3 μm.

In an embodiment, the cell trap is a rectangular prism-shaped hollow formation in the microfluidic device with an open face to permit entry of a cell into the cell trap.

Preferably, the cell trap opening is in a face that is opposite the open face.

Preferably, the cell trap has a depth that is substantially the same as the depth of the flow channel. More preferably, the cell trap has width and length dimensions that are the same as the depth. A preferred size is 20 μm×20 μm×20 μm (±5 μm).

The cell trap may include valves and/or pumps to isolate the opening connected to the flow channel inlet from the flow channel inlet and/or the opening that permits entry of a cell into the cell trap.

In an embodiment, the microfluidic device further includes chromosome dispensing structure downstream of the outlet, the chromosome dispensing structure including:

a dispensing channel defined between a channel inlet and a channel outlet, and having a port for receiving an individual chromosome from the outlet of the flow channel;

wherein the channel outlet is connected to a dispensing tube configured to dispense single chromosomes from the microfluidic device in the form of a fluid droplet including the single chromosome. In an embodiment, the dispensing channel has a depth that is the same as the depth of the flow channel.

In an embodiment, the dispensing structure further includes a chromosome holding region downstream of the series of expanded portion, the chromosome holding portion comprising an expanded region functioning to retain one or more chromosomes therein from travelling downstream whilst upstream metaphase chromosomes are still travelling through the flow channel.

In an embodiment, the microfluidic device further includes a detection zone, the detection zone comprising or associated with a device to detect the presence of metaphase chromosomes. In an embodiment, the device can detect the presence of individual metaphase chromosomes. The device may be, for example, a photodetector. Suitable outputs for detection include fluorescence.

In the detection zone, the individual metaphase chromosomes may be detected downstream of the outlet of the flow channel, where they are discretely dispensed and deposited onto a slide or well-plate for further analysis.

In a further aspect of the invention, there is provided a method for separating metaphase chromosomes in a metaphase chromosome-containing fluid, the method including:

passing the metaphase chromosome-containing fluid through the microfluidic device as defined above in one or more aspects of the invention at a pressure whereby the constrictions subject the metaphase chromosomes to sufficient shear stress to separate the metaphase chromosomes from one another.

In an embodiment of this aspect of the invention, the method further includes discretely dispensing chromosomes from the outlet.

In one form of this aspect of the invention, the metaphase chromosomes are in the form of one or more clusters of metaphase chromosomes, and wherein the constrictions subject the one or more clusters of metaphase chromosomes to sufficient shear stress to separate the one or more clusters of metaphase chromosomes into individual metaphase chromosomes; and wherein the individual metaphase chromosomes are discretely dispensed from the outlet.

In one form of this aspect of the invention, the pressure is pulsed pressure. Pulsed pressure may be applied alternately in both the forward and backward directions along the flow channel, wherein the overall pressure balance applied is such that the metaphase chromosomes incrementally move towards the outlet. Alternatively, the pressure may be constant pressure.

In a further aspect of the invention, there is provided a method for separating metaphase chromosomes in a chromosome-containing fluid, the method including:

passing a chromosome-containing fluid including metaphase chromosomes through a microfluidic device, the microfluidic device having a flow channel including:
a plurality of expanded regions located between an inlet and an outlet; and
one or more constrictions located between one or more of the expanded regions;

subjecting metaphase chromosomes, at or in the one or more constrictions, to sufficient shear stress to separate the metaphase chromosomes from one another; and

dispersing the separated metaphase chromosomes in the plurality expanded regions from one another.

In an embodiment of this aspect of the invention, the method further includes discretely discharging the separated metaphase chromosomes from the microfluidic device.

In one form of this aspect of the invention, the metaphase chromosomes are in the form of one or more clusters of metaphase chromosomes, and wherein the constrictions subject the one or more clusters of metaphase chromosomes to sufficient shear stress to separate the one or more clusters of chromosomes into individual chromosomes.

In a further aspect of the invention, there is provided a method for separating metaphase chromosomes in a chromosome-containing fluid with a microfluidic device, the method including:

passing the fluid through a flow channel of a microfluidic device, the flow channel having a plurality of alternating constrictions and expansions;

wherein when the fluid is passed through a constriction, the method includes applying a pressure pulse to subject the metaphase chromosomes to a shear stress sufficient to separate the metaphase chromosomes from one another;

wherein when the fluid is passed through an expansion, the microfluidic device is operated at a pressure to disperse the separated chromosomes from one another.

In an embodiment of this aspect of the invention, the method further includes discretely discharging the separated chromosomes from the microfluidic device.

In one form of this aspect of the invention, the metaphase chromosomes are in the form of one or more clusters of metaphase chromosomes, and wherein when the fluid is passed through a constriction, the pressure pulse subjects the one or more clusters of metaphase chromosomes to a shear stress sufficient to separate metaphase chromosomes from the one or more clusters of metaphase chromosomes.

In accordance with various of the above-mentioned aspects of the invention, the shear stress may be from at least about 0.02 N/m²to at least about 15,000 N/m² or any value in between as measured at walls of the minimum width of a constriction. Preferably, the shear stress is from about 0.02 N/m²to about 12,500 N/m², about 0.02 N/m²to about 10,000 N/m², about 0.02 N/m²to about 9,000 N/m², about 0.02 N/m²to about 8,500 N/m², about 0.02 N/m²to about 8,000 N/m², about 0.02 N/m²to about 7,000 N/m², about 0.02 N/m² to about 6,000 N/m², about 0.02 N/m² to about 5,000 N/m², about 0.02 N/m²to about 4,000 N/m², about 0.02 N/m²to about 3,500 N/m², about 0.02 N/m² to about 3,000 N/m², about 0.02 N/m²to about 2,500 N/m², about 0.02 N/m²to about 2,000 N/m², about 0.02 N/m² to about 1,500 N/m², about 0.02 N/m² to about 1,000 N/m², about 0.02 N/m²to about 800 N/m², about 0.02 N/m²to about 600 N/m², about 0.02 N/m²to about 500 N/m², about 0.02 N/m²to about 400 N/m², about 0.02 N/m²to about 300 N/m², about 0.02 N/m² to about 250 N/m², about 0.02 N/m² to about 200 N/m², about 0.02 N/m² to about 150 N/m², about 0.02 N/m²to about 100 N/m², about 0.02 N/m² to about 80 N/m², about 0.02 N/m²to about 60 N/m², about 0.02 N/m² to about 50 N/m², about 0.02 N/m² to about 40 N/m², about 0.02 N/m²to about 30 N/m², about 0.02 N/m² to about 25 N/m², about 0.02 N/m²to about 10 N/m², about 0.02 N/m² to about 5 N/m², or about 0.02 N/m² to about 1 N/m², as measured at walls of the minimum width of a constriction. Preferably, the shear stress is from about 1 N/m²to about 15,000 N/m², about 2 N/m²to about 12,500 N/m², about 5 N/m²to about 10,000 N/m², about 5 N/m² to about 9,000 N/m², about 10 N/m²to about 8,500 N/m², about 10 N/m² to about 8,000 N/m², about 15 N/m² to about 7,000 N/m², about 15 N/m² to about 6,000 N/m², about 20 N/m²to about 5,000 N/m², about 20 N/m²to about 4000 N/m², from about 25 N/m² to about 4,000 N/m², about 50 N/m²to about 3,000 N/m², about 100 N/m² to about 2,000 N/m², about 200 N/m² to about 1,000 N/m², about 100 N/m² to about 500 N/m², about 200 N/m² to about 400 N/m², about 5 N/m² to about 500 N/m², about 10 N/m² to about 400 N/m², about 20 N/m² to about 100 N/m², or about 30 N/m² to about 60 N/m², as measured at walls of the minimum width of a constriction. Preferably, the shear stress is greater than about 0.2 N/m², about 1 N/m², about 5 N/m², about 10 N/m², about 25 N/m², about 30 N/m², about 40 N/m², about 50 N/m², about 60 N/m², about 80 N/m², about 100 N/m², about 150 N/m², about 200 N/m², about 250 N/m², about 300 N/m², about 400 N/m², about 500 N/m², about 600 N/m², about 800 N/m², about 1,000 N/m², about 1,500 N/m², about 2,000 N/m², about 2,500 N/m², about 3,000 N/m², about 3,500 N/m², about 4,000 N/m², about 5,000 N/m², about 6,000 N/m², about 7,000 N/m², about 8,000 N/m², about 8,500 N/m², about 9,000 N/m², about 10,000 N/m², or about 12,500 N/m², as measured at walls of the minimum width of a constriction. Preferably, the shear stress is less than about 1 N/m², about 5 N/m², about 10 N/m², about 25 N/m², about 30 N/m², about 40 N/m², about 50 N/m², about 60 N/m², about 80 N/m², about 100 N/m², about 150 N/m², about 200 N/m², about 250 N/m², about 300 N/m², about 400 N/m², about 500 N/m², about 600 N/m², about 800 N/m², about 1,000 N/m², about 1,500 N/m², about 2,000 N/m², about 2,500 N/m², about 3,000 N/m², about 3,500 N/m², about 4,000 N/m², about 5,000 N/m², about 6,000 N/m², about 7,000 N/m², about 8,000 N/m², about 8,500 N/m², about 9,000 N/m², about 10,000 N/m², about 12,500 N/m², or about 15,000 N/m², as measured at walls of the minimum width of a constriction.

In accordance with various of the above-mentioned aspects of the invention, a pressure is preferably applied across the flow channel. The pressure applied across the flow channel is from about 0 mbar to about 10,000 mbar or any value in between. Preferably, the pressure applied is from about 2 mbar to about 7,000 mbar, about 30 mbar to about 5,000 mbar, about 50 mbar to about 2,500 mbar, about 100 mbar to about 1,000 mbar, about 250 mbar to about 1,000 mbar, about 300 mbar to about 1,000 mbar, or about 400 mbar to about 700 mbar. Preferably, the pressure applied is greater than about 0 mbar, about 2 mbar, about 30 mbar, about 50 mbar, about 100 mbar, about 250 mbar, about 300 mbar, about 400 mbar, about 700 mbar, about 1,000 mbar, about 2,500 mbar, about 5,000 mbar, or about 7,000 mbar. Preferably, the pressure applied is less than about 2 mbar, about 30 mbar, about 50 mbar, about 100 mbar, about 250 mbar, about 300 mbar, about 400 mbar, about 700 mbar, about 1,000 mbar, about 2,500 mbar, about 5,000 mbar, about 7,000 mbar, or about 10,000 mbar.

In accordance with various of the above-mentioned aspects of the invention, the method may include:

trapping a metaphase cell in a cell trap of the microfluidic device; and

introducing a lysis buffer to the metaphase cell and applying a pressure pulse to drive the metaphase cell from the cell trap and into the flow channel under sufficient shear stress to lyse the cell and provide the metaphase chromosomes in the chromosome-containing fluid.

In various of the above-mentioned aspects of the invention, the method may further include:

receiving the dispensed individual chromosomes from the outlet of the flow channel into a dispensing channel of the microfluidic device;

transporting the individual chromosomes to a dispensing tube; and

dispensing single chromosomes from the microfluidic device via the dispensing tube in the form of a fluid droplet including the single chromosome.

Preferably, the fluid droplet has a volume of from about 100 nL up to about 500 nL. More preferably from about 100 nL up to about 400 nL. Even more preferably, 100 nL up to about 300 nL.

In one or more of the aspects described above, and embodiments thereof, the chromosome-containing fluid includes a lysis buffer such that the method is a method for the chemically-assisted shear separation of metaphase chromosomes.

As will be understood, a lysis buffer is a buffer that aids lysis of a cell. A separation buffer is a buffer that aids the separation of chromosomes. A lysis buffer can include a separation buffer, and vice versa.

In one or more of the aspects described above, and embodiments thereof, a lysis buffer is introduced such that the cell is lysed through the chemical action of the lysis buffer or a combination of chemical and physical action. Incorporated in the lysis buffer or subsequent to the lysis buffer a separation buffer is introduced before or with introduction of the metaphase chromosomes in the chromosome-containing fluid to the inlet of the flow channel.

In one or more of the aspects of the invention described above, and embodiments thereof, a chromosome specific label; and/or a DNA stain may be added prior to the introduction of the cells into the inlet of the device. In one or more of the aspects of the invention described above, and embodiments thereof, the metaphase cells may be fixed and permeabilised to facilitate the hybridisation of a chromosome specific label; and/or a DNA stain prior to the introduction of the cells into the inlet of the device.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic of a microfluidic device according to one embodiment of the invention.

FIG. 2: Schematic of the microfluidic device illustrating the constriction.

FIG. 3: Schematic of the microfluidic device illustrating the sampling port through which cells are introduced into the microfluidic device.

FIG. 4: Schematic of the microfluidic device illustrating the upstream cell trap and lysing structure.

FIG. 5: Schematic of the microfluidic device illustrating the chemically assisted shear lysing of the cell and transfer under pressure pulse from the cell trap and into the flow channel.

FIG. 6: Schematic of the microfluidic device illustrating the flow channel and expanded regions.

FIG. 7: Schematic of the microfluidic device illustrating the flow channel outlet and chromosome detection.

FIG. 8: Schematic of the microfluidic device illustrating downstream chromosome isolation for individual chromosome dispensing.

FIG. 9: Schematic illustrating the dispensing of a droplet containing a single chromosome from the microfluidic device onto a well plate.

FIG. 10: Schematic showing the pump arrangement of the microfluidic device.

FIG. 11: Close up drawing showing detail of a constriction within the flow channel of the microfluidic device.

### REFERENCES

Quake et al. (Nature Methods, Vol. 11, No. 1, 2014, pp 19-21)

Dolez̆el et al. (Funct. Integr. Genomics, 2012, 12:397-416)

Fan et al. (Nat. Biotechnol., January 2011, 29(1):51-57)

### DETAILED DESCRIPTION OF THE EMBODIMENTS

The present invention relates to a microfluidic device and method for separating metaphase chromosomes from one another.

In a preferred form, the microfluidic device is configured to trap and lyse a single metaphase cell, suspend the expelled chromosomes into singulated chromosomes, detect each singulated chromosome, and then dispense each chromosome from the microfluidic device onto a receptacle (such as a glass slide or a well plate) for post processing.

Broadly, a cell is introduced into the microfluidic device where it is analysed (such as via optical microscopy) to determine whether the cell is a metaphase cell. If the cell is a metaphase cell it is trapped, then a lysis buffer is introduced into the microfluidic device accompanied with a high pressure pulse to drive the cell and its contents from the cell trap through a channel restriction and into a flow channel of the microfluidic device whilst lysing the cell via shearing on the cell membrane as it passes through the channel restriction. The chromosomes, typically in the form of one or more clusters, then emerge into the microfluidic flow channel. In the microfluidic flow channel, the one or more clusters of chromosomes are passed through an alternating series of constrictions and expansions.

The constrictions provide an impediment to the flow of the one or more clusters of chromosomes through the flow channel. A pressure pulse drives the one or more clusters of chromosomes through the constrictions, which at the same time, applies significant shear stress to the one or more clusters of chromosomes to break the one or more clusters apart.

In the expansions, the chromosomes are subjected to a lower flow velocity and varying flow profiles which permits the chromosomes to disperse and become separated from one another. The expansions also provide the lysis buffer with an opportunity to mix with the individual chromosomes to stabilise those chromosomes, and to mix with the one or more clusters of chromosomes to chemically assist in the shear separation of chromosomes in subsequent constrictions.

The one or more clusters of chromosomes are subjected to multiple alternating constrictions and expansions until the one or more clusters of chromosomes have been broken down into separate and individual chromosomes. These individual chromosomes are detected at the outlet of the flow channel, where they are discretely dispensed and deposited onto a slide or well-plate for further analysis.

In this way, the device and method of the invention provides a mechanism for separating individual chromosomes for subsequent haplotype determination.

An embodiment of the invention is described below.

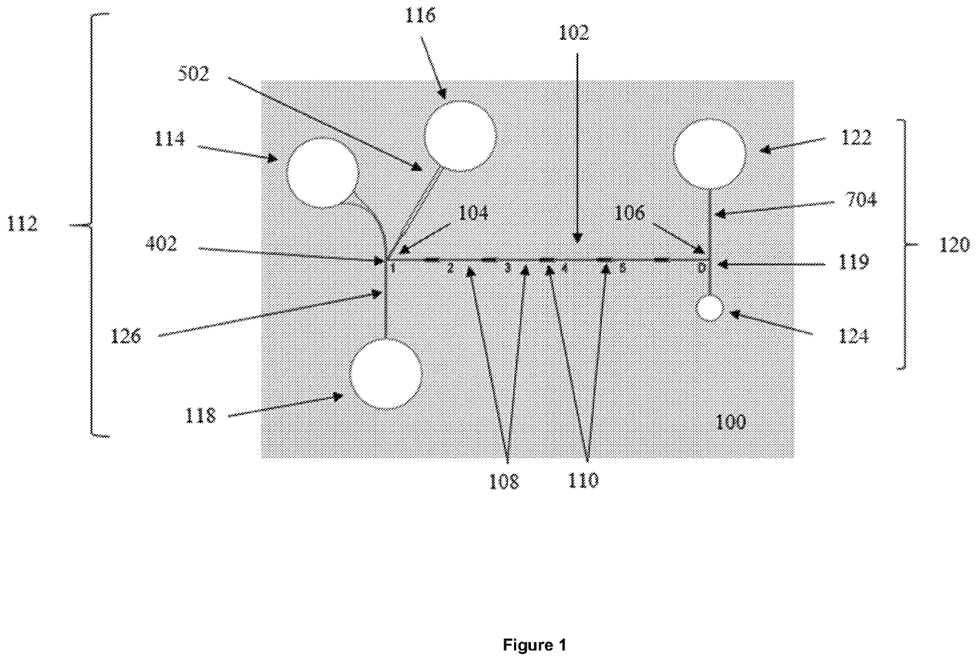

FIG. 1 is a schematic of a microfluidic device 100 for separating and dispensing single chromosomes from a chromosome suspension. In this case, the microfluidic device 100 is formed as a polydimethylsiloxane (PDMS) casting on wafer tooling. The skilled addressee will appreciate that a number of different materials may be used. The PDMS casting is capped with a glass cover slip. Again, different materials may be used. However, glass was selected as the capping material due to its optical properties (e.g. optical clarity and no autofluorescence) readily permitting observation of the components of the microfluidic device 100 and its ability to bond with PDMS via plasma activation.

The microfluidic device 100 includes a microfluidic flow channel 102 having an inlet 104 and an outlet 106. In this embodiment, the flow channel 102 has a length of 5 mm. However, different lengths could be used, such as from 3 mm to 15 mm. The flow channel 102 is divided into five zones (labelled as 1 to 5 in FIG. 1). Each of these zones includes a first flow channel portion 108 and a second flow channel portion representing an expanded portion 110. The first flow channel portion 108 has a cross-sectional area transverse to the flow direction that is less than the cross-sectional area of the expanded portion 110. In this particular case, the flow channel 102 has a depth of 20 μm, the first channel portion 108 has a width of 20 μm (e.g. a cross-sectional flow area of 400 μm²), and the expanded portion 110 has a width of 100 μm (e.g. a cross-sectional flow area of 2000 μm²). Thus, in the present embodiment, the ratio of the cross-section flow are of the first flow channel portion 108 to the expanded portion 110 is 1:5. Although this embodiment has a ratio of 1:5, the inventors are of the view that a ratio of from 1:2 to 1:10 is suitable.

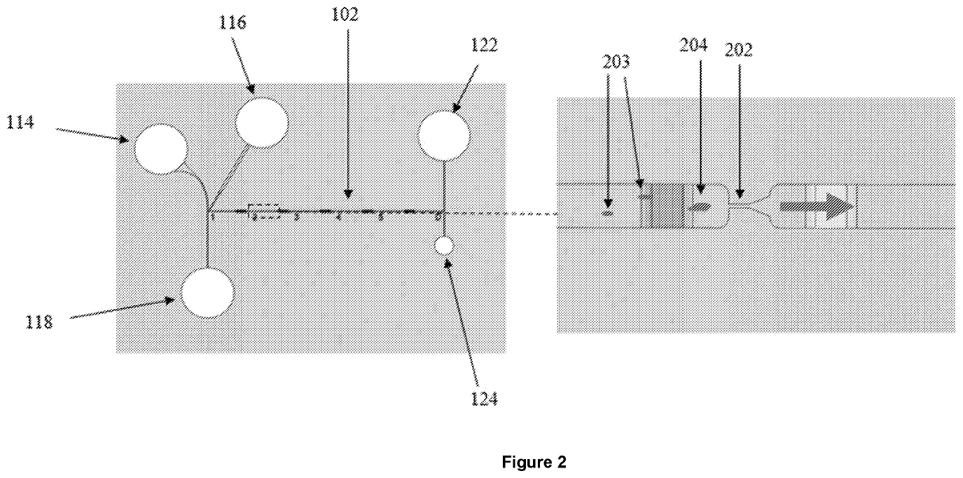

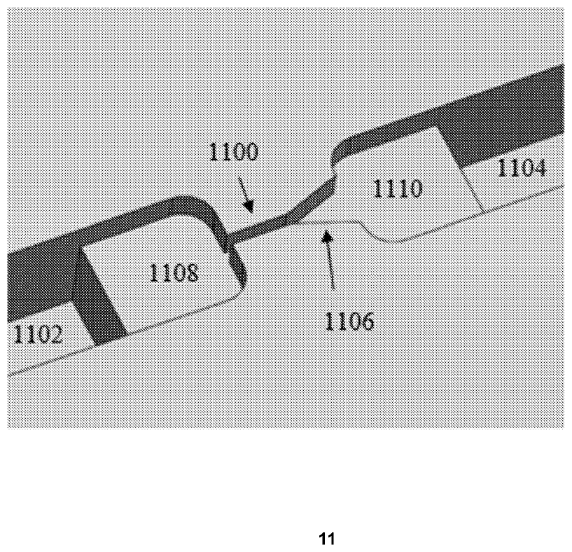

Each of the first flow channel portions 108 includes a constriction 202 (see FIG. 2 and FIG. 11, expanded view). It will be appreciated that each of the first flow channel portions 108 may include multiple constrictions 202. In this embodiment, the constrictions 202 have a width of from about 1 μm to about 2 μm. Furthermore, the width of the constrictions 202 in each successive first flow channel portions 108 from the inlet 104 to the outlet 106 is less than the width of the constrictions 202 in preceding first flow channel portions 108.

FIG. 11 illustrates an embodiment of a constriction 1100 between flow channel portions 1102 and 1104. The constriction 1100 has widened tapered outlet 1106 that tapers to a width that is approximately half the width of the flow channel. In FIG. 11, the constriction has a depth that is less than flow channel portions 1102 and 1104. In this particular embodiment, the constriction has a depth of about 5 μm whereas the flow channel portions 1102 and 1104 have a bulk depth of about 20 μm. Regions of the flow channel immediately adjacent to the constriction 1100, labelled as items 1108 and 1110, have the same depth as the constriction (e.g. about 5 μm) such that there is a step change in depth within the flow channel from the depth of the constriction 1100 (e.g. about 5 μm) to the bulk depth of the flow channel (e.g. about 20 μm).

The operation of the components of the flow channel 102 will now be briefly described. During operation, a fluid including one or more clusters of chromosomes is introduced under pressure into the flow channel 102 via inlet 104. The fluid flows through the first flow channel portion 108 of zone 1 where it passes through a constriction 202. The constriction 202 impedes the passage of the one or more clusters of chromosomes therethrough. The approximate size of a single metaphase chromosome is from about 0.5 μm to about 3 μm; whereas a chromosome cluster can range in size from slightly larger than a single metaphase chromosome to slightly less than the size of the metaphase cell (approx. 10 μm-15 μm). In any event, fluid in the constriction is subject to increased flow velocity relative to the flow channel 102 by virtue of providing a narrow flow area, and this increased flow velocity forces the one or more clusters of chromosomes through the restriction while subjecting the one or more clusters of chromosomes to substantial shear stress such as around 0.02 N/m² to about 1 N/m² at the walls depending on the dimensions of the constriction, pressures applied (which in turn effects velocity) and fluid properties. This shear stress is sufficient to fragment one or more clusters of chromosomes which can result in single chromosomes being dislodged from the one or more clusters of chromosomes breaking apart into smaller chromosome clusters. The single chromosomes 203 and/or smaller chromosome clusters 204 then emerge via a widening tapered outlet of the constriction 202 of the first flow channel portion 108 of the flow channel 102 downstream of the constriction 202 before passing into the second channel portion, e.g. the expanded portion 110, of zone 1. In the expanded portion 110, the single chromosomes and/or smaller chromosome clusters are subjected to reduced flow velocity relative to the first flow channel portion 108 by virtue of the wider flow area. In this expanded portion 110, the single chromosomes and/or smaller chromosome clusters disperse in both the radial and axial directions via a combination of diffusion and advection which can result in increased spacing between the chromosomes when they exit the expanded portion to the narrower first flow channel portion 108 of zone 2. Additionally, the dispersal of chromosomes and/or smaller chromosome clusters in the expanded region 110 allows reagents that may be present in the chromosome containing fluid to mix and diffuse around the surface of the chromosomes and/or smaller chromosome clusters (e.g. stabilisers or other reagents that promote separation of the chromosomes and/or prevent or minimise aggregation).

In zone 2, the single chromosomes 203 and/or smaller chromosome clusters 204 undergo a similar process in that they pass through a first flow channel portion 108 having a constriction 202. However, in this case the constriction 202 in Zone 2 is narrower than the constriction 202 in Zone 1. The reason for this is to impede the passage of the smaller chromosome clusters, and to provide a higher flow velocity to subject the smaller chromosome clusters to higher shear stresses to further break apart the chromosome clusters and/or separate single chromosomes from the chromosome clusters. Again, after passing through this constriction, the chromosomes similarly emerge into the first flow channel portion 108 of Zone 2, before passing into the expanded region 110 of Zone 2 for further dispersal.

The above process is repeated through Zones 3, 4, and 5 whereby each constriction 202 in the first flow portion 110 of these zones decreases in width to impede the passage of and break apart smaller chromosome clusters 204; and each expanded region 110 of these zones further disperses single chromosomes 203 and/or chromosome clusters 204 from one another.

After passing through each of the zones of the flow channel 102, chromosomes then pass through the outlet 106 of the flow channel as single chromosomes spaced axially apart from one another. Because the single chromosomes are spaced axially apart, the single chromosomes can be isolated from one another for downstream purposes.

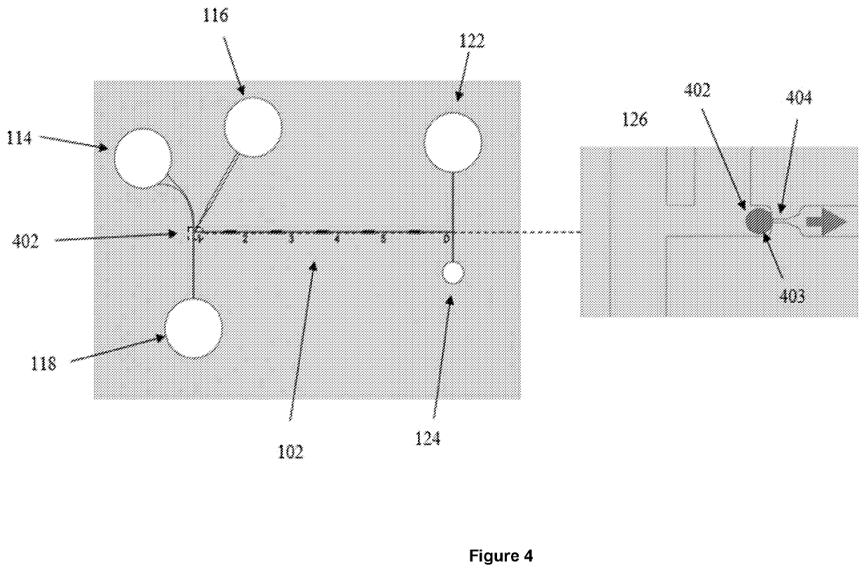

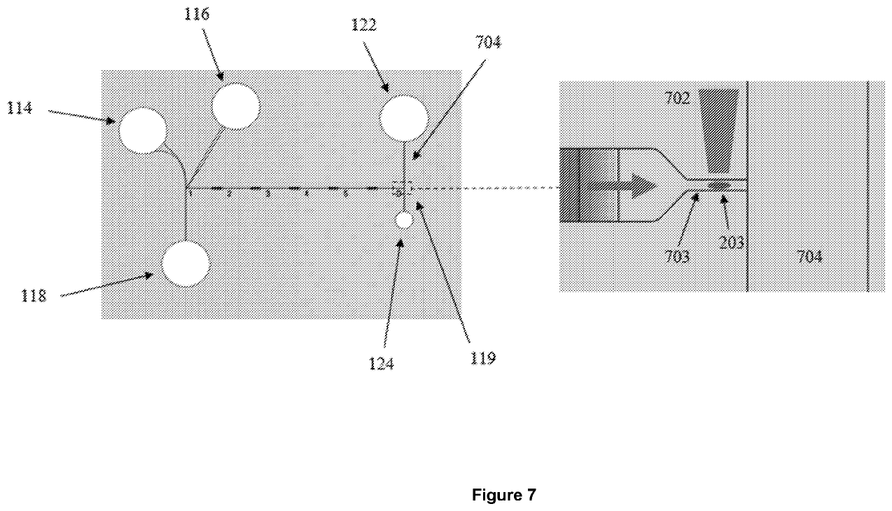

In the embodiment depicted in FIG. 1 the microfluidic device 100 includes a cell capture and lysis structure 112 upstream of the flow channel 102. The cell capture and lysis structure 112 includes a sample port 114 for introducing a fluid containing cells, a cell trap 402 (see FIG. 4, expanded view) for trapping a cell to permit interrogation of the cell, a lysis port 116 for introducing a lysis buffer to lyse the cell and release chromosomes contained therein if the cell is deemed suitable, and a waste port 118 for discharging waste reagents and cells that are deemed unsuitable. At the outlet of the flow channel, the device includes a detection zone 119 (see FIG. 7, expanded view) for detecting individual metaphase chromosomes to ensure that the chromosomes are dispensed. Downstream of the flow channel 102, the microfluidic device includes a dispensing structure 120 including a dispensing port 122, an extraction port 124 and a dispense channel 704. The cell trap 402 has dimensions of 20 μm×20 μm×20 μm which is sufficiently small to hold a single metaphase cell. The cell trap 402 includes an opening 404 to the inlet of flow channel 102. The opening 404 has a width of about 2 μm to about 3 μm to prevent a cell from passing from the cell trap 402 into the flow channel 102.

During operation, a cell sample can be provided to the microfluidic device via sample port 114.

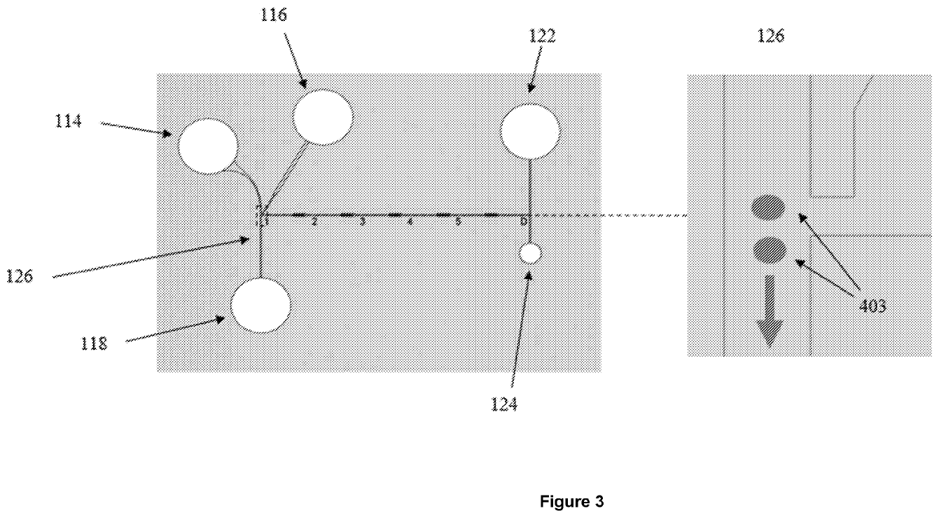

FIG. 3 shows the addition of a fluid sample containing cells 403 via the sample port 114. In FIG. 3, the sample port 114 is operated at high pressure, the waste port 118 is operated at low pressure, the lysis port 116 and dispensing port 122 are operated at datum pressure, and the extraction port 124 is closed. Given this arrangement, the sample flows through sample transfer channel 126 to waste port 118.

FIG. 4 illustrates the capture of a cell 403 in the cell trap 402 for interrogation. In FIG. 4 the sample port 114, lysis port 116, and waste port 118 are operated at datum pressure; the dispensing port 122 is operated at low pressure; and the extraction port 124 is closed. The effect of this arrangement is to provide a pressure differential that maintains the cell 403 in the cell trap, e.g. there is a suction effect that biases the cell in the cell trap 402 against opening 404. However, the cell 403 is unable to pass through opening 404. Once the cell 403 is held in the cell trap 402, visual inspection is possible through the glass coverslip. The purpose of the visual inspection is to confirm that the cell 403 is a metaphase cell—and thus suitable for obtaining a chromosome suspension.

If the cell 403 is not a metaphase cell, then the cell 403 is flushed from the cell trap 402, such as by applying a back pressure via the dispensing port 122 and discharging the cell through the waste port 118. That is, the dispensing port 122 is operated at high pressure; the waste port 118 is operated at low pressure, the sample port 114 and the lysis port 116 are operated at datum pressure; and the extraction port 124 is closed.

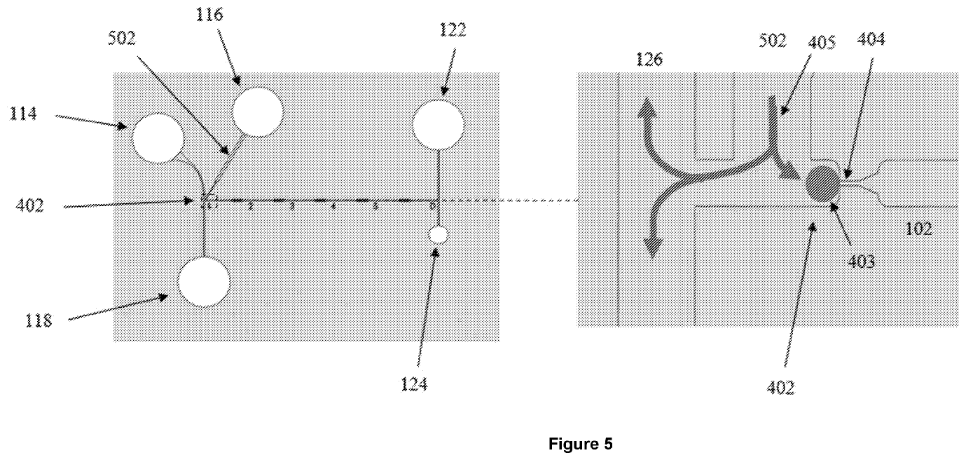

If the cell is a metaphase cell, then the cell is subjected to a lysing process to rupture the cell membrane and release the chromosomes from within the cell. This process is shown in FIG. 5. In FIG. 5, lysis buffer 405 is applied by operating the lysis port 116 at a higher pressure (e.g. 40-45 mbar), the dispensing port 122 is operated at low pressure (e.g. below the datum pressure of 35 mbar, such as less than 30 mbar); the sample port 114 and the waste port 118 are operated at datum pressure (e.g. 35 mbar); and the extraction port 124 is closed. The effect of this is that a lysis buffer 405 flows from the lysis port 116 through the lysis channel 502 where it contacts the cell to be lysed.

A pressure pulse is then used to force the cell through the opening and along a constriction which lyses the cell by shearing the cell membrane, and passing the contents of the cell (including one or more clusters of chromosome) into the flow channel 102 via the inlet 104. In this pressure configuration, the sample port 114, the wasteport 118, and the lysis port 116 are operated under a pulse pressure; with the dispense port 122 being operated at low pressure and the extraction port 124 being closed.

The lysis buffer is an aqueous solution that can include Type 1 ultrapure water, 2 v/v % acetic acid, 5 w/v % triton X-100 also known as Polyethylene glycol p-(1,1,3,3-tetramethylbutyl)-phenyl ether (a non-ionic surfactant that has a hydrophilic polyethylene oxide chain (on average it has 9.5 ethylene oxide units) and an aromatic hydrocarbon lipophilic or hydrophobic group), 0.1 w/v % pepsin, 75 mM potassium chloride. In this buffer; the acetic acid fixes and preserves the chromosome morphology, the triton X-100 solubilise/lyse the cell membrane components and the hydrophobic proteins and has a secondary role in releasing chromosomes, the pepsin releases individual chromosomes from their clusters and aids cell lysis and removes cellular proteins, and the potassium chloride is a salt used to swell the cells via osmotic pressure and enhances pepsin solubility. Alternatively, the buffer may include 0.1% w/v pepsin, 1 mM EDTA, 73 mM potassium acetate buffer, 2 mM magnesium sulphate, buffered to pH 5 with acetic acid. Alternatively, the fixative role of acetic acid in either of these buffers could be performed by fixative formaldehyde. A person skilled in the art would appreciate that other buffer compositions known in the art would also be suitable for use as the lysis and/or separation buffer.

FIG. 2 illustrates the use of a pressure pulse to drive chromosome clusters through the constrictions 202. This is achieved by applying a high pressure pulse through the lysis port 116 (e.g. 300-1000 mbar). Under this mode of operation, the sample port 114 and the waste port 118 are operated at a datum pressure (e.g. 35 mBar); the dispensing port 122 is operated at low pressure (e.g. <30 mbar); and the extraction port 124 is closed. The additional pressure from the lysis port 118 drives impeded clustered chromosomes 204 (e.g. one or more clusters of chromosomes that may have become trapped at the narrow opening of the constriction 202) through the constriction 202 subjecting the one or more clusters of chromosomes to high shear stress conditions to break the one or more clusters of chromosomes 204 into single chromosomes 203 and/or smaller chromosome clusters. This process is repeated for the various zones.

An alternative approach is to apply a high pressure pulse via the sample port 114 (e.g. 250-950 mBar or 250-1,000 mBar), the waste port 118 (e.g. 250-950 mBar or 250-1000 mBar), and the lysis port 116 (e.g. 300-1000 mBar); low pressure at the dispensing port 122 (e.g. 0 mBar); and the extraction port 124 is closed.

Under the pressure regimes described, the shear stress through the constriction zones range from about 0.02 N/m² to 15,000 N/m² depending on the dimensions of the constriction and pressures applied.

The combination of the lysis buffer and the pressure differential between the lysis port 116 and the dispensing port 122 induces a chemically-assisted shear lysing process which causes the cell membrane to rupture and forces the contents of the cell through opening 404 and into the flow channel 102. The contents of the cell include one or more clusters of chromosomes 204 (and potentially single chromosomes 203). The one or more clusters of chromosomes are then subjected to the shear treatment process in channel 102 as hereinbefore described to separate the chromosomes.

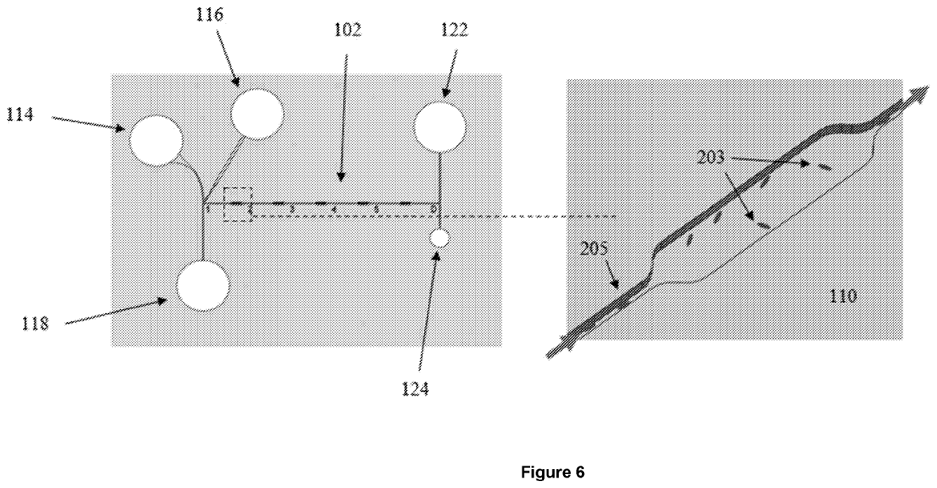

FIG. 6 provides an illustration of the operation of the microfluidic device 100 after the cell has been lysed and the chromosomes 205 expelled into the flow channel 102 and moving through the expanded region 110 of one of the zones. In FIG. 6 the sample port 114, lysis port 116, and waste port 118 maintain the lysis buffer application settings; the dispensing port 122 is operated at datum pressure; and the extraction port 124 is closed. Thus, a pressure differential exists across the flow channel 102 that drives the chromosomes from the inlet 104 of the flow channel 102 toward the outlet 106 of the flow channel. The expanded section shows single chromosomes 203 dispersing and being separated through the expanded region 110 of zone 1. The expanded region 110 may include a mixing apparatus, such as a herringbone mixer to assist the dispersal of the single chromosomes.

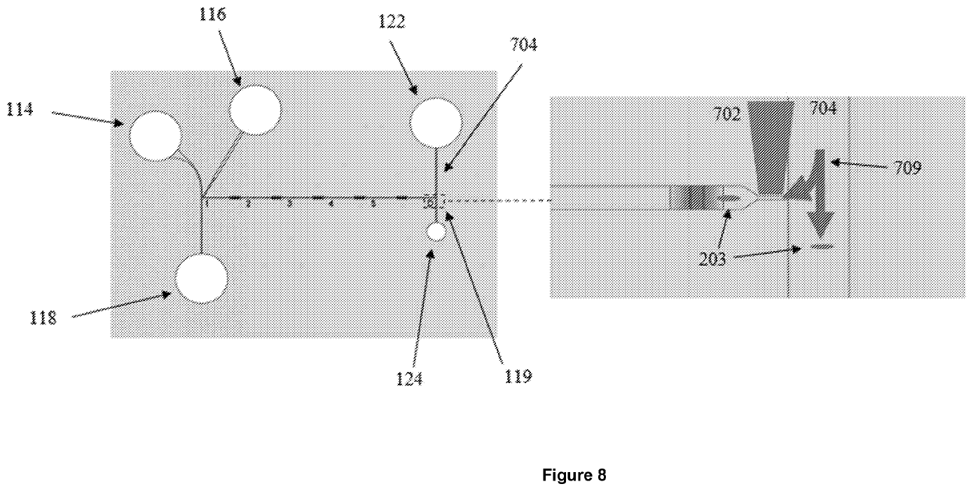

Once the chromosomes are separated they are detected at the outlet 106, such as by live recording of fluorescent signals. Each detection event triggers the dispense system to activate. FIGS. 7 and FIG. 8 illustrate the detection and count of chromosomes, and the transfer of single chromosomes out of the microfluidic device 100 via extraction port 124. FIG. 7 illustrates the detection of a single chromosome 203 at the outlet 106 using a photodetector 702. The detection restriction 703 ensures that chromosomes are in single file. On detection of a chromosome, the dispensing system is activated. A flow of neutralisation buffer (to stop the degradation of chromosome morphology from Pepsin activity, if present) is provided via dispensing port 122 to capture the detected chromosome and dispense it from the microfluidic device 100. Each chromosome is discharged from the microfluidic device in the form of a droplet. The droplet is dispensed onto a receptacle (e.g. a glass slide or specialised well plate). In more detail, once the chromosome is detected, a valve on the extraction port 124 is switched from the closed position (shown in FIG. 7) to the open position and the pressure of the dispensing port 122 is increased to provide the neutralisation agent. This increased flow 709 (shown in FIG. 8) results in the single chromosome 203 being dispensed from the outlet 106 and into dispensing channel 704 where it is subsequently deposited onto a well plate or glass slide. The increased flow 709 also results in a flow reversal in the flow channel 102, helping to keep the chromosomes separated.

By way of example, during detection the sample port 116 and waste port 118 are operated at 0 mBar; the lysis port 116 is operated at from 2-5 mBar; and the dispensing port 122 is operated at 2 mBar. As an alternative example, during detection the sample port 116 and waste port 118 are operated at 10 mBar; the lysis port 116 is operated at at 20 mBar; and the dispensing port 122 is operated at 2 mBar This low pressure differential slows the flow through the flow channel 102 to permit detection of chromosomes at the outlet 106. Once a chromosome has been detected the pressure at the dispensing port 122 is increased to 15 mBar to dispense the chromosome from the outlet 106 and into the dispensing channel 704.

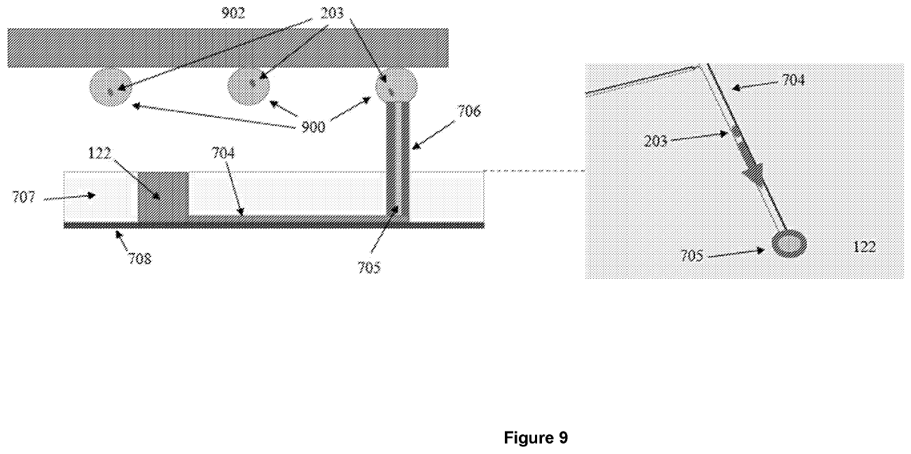

FIG. 9 illustrates the deposition of a 200 nL droplet 900 including a single chromosome 203 from an outlet of dispensing channel 704 onto a moving well plate 902 through dispense tube 705. This process may be repeated until each chromosome has been deposited onto the well plate 902 such as in an array, e.g. for chromosomes taken from a human cell, there will be 46 discrete droplets each including a single chromosome. The hydrophobic coating 706 of the dispense tube ensures that the droplet does not stick to the dispense tube 705. FIG. 9 also illustrates the dispense channel 704 in relation to the cartridge 707 and the glass coverslip 708.

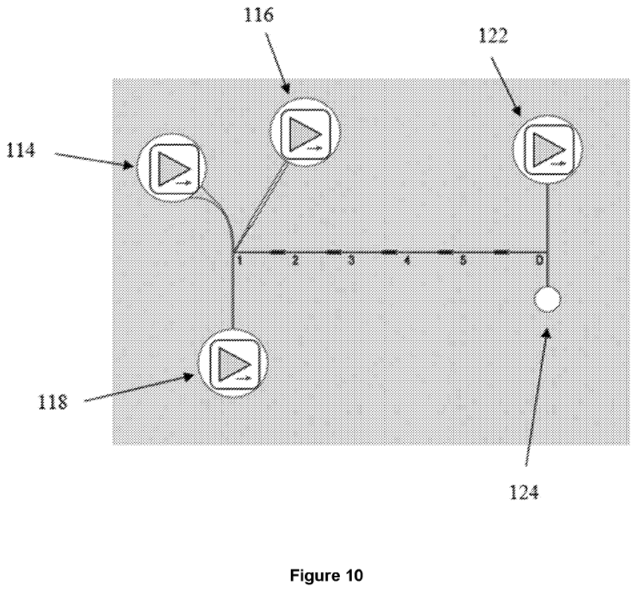

FIG. 10 shows the pump arrangement according to one embodiment of the invention with datum pressures. In this embodiment, the sample port 114 is configured to use a 69 mBar pressure pump with the datum pressure set to 35 mBar; the lysis port 116 is configured to use a 1000 mBar pressure pump with the datum pressure set to 35 mBar; the waste port 118 is configured to use a 70 mBar pressure pump with the datum pressure set to 35 mBar; the dispensing port 122 is configured to use a 345 mBar pressure pump with the datum pressure set to 35 mBar; and the extraction port 124 is normally closed.

As generally described above, operation of the microfluidic device 100 is carried out by connecting the various fluid ports of the microfluidic device to pressure/flow controllers. The 345 mbar pressure pumps are connected to the sample port 114 and the waste port 118 because they are used to control cell motion during cell screening and trapping, which requires high resolution in pressure change to generate and maintain low flow rates. One 1000 mBar pressure pump is connected to the lysis port 116 to provide high pressure pulses to induce shear in the cell that is held in the trap. A 69 mBar is connected to the dispensing port 122 to allow pressure drop in the dispense channel for chromosome transfer. The dispensing channel 704 will have a valve (seated tube on a gasket) on the extraction port 124 that will normally be closed during operation except when dispensing droplets. All the pressure controllers will initially be set to a datum pressure of 35 mBar, from this datum pressure, each pressure line can either be raised or dropped depending on the required direction of flow within the microfluidic device 100.

Alternatively, in this embodiment, the sample port 114 is configured to use a 1000 mBar pressure pump with the datum pressure set to 35 mBar; the lysis port 116 is configured to use a 1000 mBar pressure pump with the datum pressure set to 35 mBar; the waste port 118 is configured to use a 1000 mBar pressure pump with the datum pressure set to 35 mBar; the dispensing port 122 is configured to use a 345 mBar pressure pump with the datum pressure set to 35 mBar; and the extraction port 124 is normally closed.

As generally described above, operation of the microfluidic device 100 is carried out by connecting the various fluid ports of the microfluidic device to pressure/flow controllers. The 1000 mbar pressure pumps are connected to the sample port 114 and the waste port 118 because they are used to control cell motion during cell screening and trapping, which requires high resolution in pressure change to generate and maintain low flow rates. One 1000 mBar pressure pump is connected to the lysis port 116 to provide high pressure pulses to induce shear in the cell that is held in the trap. A 345 mBar is connected to the dispensing port 122 to allow pressure drop in the dispense channel for chromosome transfer. The dispensing channel 704 will have a valve (seated tube on a gasket) on the extraction port 124 that will normally be closed during operation except when dispensing droplets. All the pressure controllers will initially be set to a datum pressure of 35 mBar, from this datum pressure, each pressure line can either be raised or dropped depending on the required direction of flow within the microfluidic device 100.

Dispensing is conducted by dispensing droplets from the microfluidic device 100 via a dispensing tube 705 (for example, the dispensing tube of the present embodiment has an outer diameter 0.79 mm, an inner diameter 0.15 mm, and a length of 7 mm), where each droplet contains a chromosome. This is done by creating a higher pressure at the dispensing port 122 and opening the valve at the extraction port 124. Fluid then travels due to the pressure drop through the dispensing channel 704, through the dispensing tube, and out of a dispensing tube tip. Once a correct droplet size is generated (droplet size is varied via varying the pressure drop but an example size is 200 nL), each droplet will be dispensed onto a glass slide or a specially designed well plate. The pressure from the dispensing port 122 will then return to datum pressure. The droplet attaches to the receptacle by the surface tension of the formed droplet. To dispense each droplet in an array and onto the receptacle, an automated mechanism that holds the receptacle is utilised. The mechanism moves independently to the cartridge in three axes, such as along x and y axes to create the array of droplets on the receptacle and in the z axis to attach each droplet to the receptacle.

It will be understood that the invention disclosed and defined in this specification extends to all alternative combinations of two or more of the individual features mentioned or evident from the text or drawings. All of these different combinations constitute various alternative aspects of the invention. For instance, it will be understood that alternative topologies of the individual features described above constitute alternative aspects of the invention.

1. A microfluidic device for separating metaphase chromosomes in a metaphase chromosome-containing fluid, the microfluidic device including:
a flow channel including:
an inlet to receive a fluid including metaphase chromosomes;
an outlet to discretely dispense individual metaphase chromosomes;
a series of expanded regions; and
one or more constrictions located between consecutive expanded regions in the series of expanded regions;
wherein the constrictions are operable to apply sufficient shear stress to separate the metaphase chromosomes from one another; and
the expanded regions are operable to disperse chromosomes from one another.

2. A microfluidic device for separating metaphase chromosomes in a metaphase chromosome-containing fluid, the microfluidic device including:
a flow channel having a width of from about 10 μm to about 30 μm, the flow channel including:
an inlet;
an outlet; and
a series of expanded regions, and one or more constrictions located between consecutive expanded regions in the series of expanded regions;
wherein the plurality of expanded regions have a channel width of from about 50 μm to about 150 μm, and each constriction in the plurality of constrictions has a minimum width of from about 1 μm to about 3 μm.

3. The microfluidic device of claim 1 or claim 2, wherein the flow channel has a depth of from about 5 μm up to about 40 μm.

4. The microfluidic device of any one of the preceding claims, wherein the length of the flow channel is from about 2 mm to about 15 mm.

5. The microfluidic device of any one of the preceding claims, wherein each successive constriction from the inlet to the outlet has a smaller minimum width than a preceding constriction.

6. The microfluidic device of any one of the preceding claims, wherein one or more of the one or more constrictions has a widening tapered outlet.

7. The microfluidic device of any one of the preceding claims, wherein each of the expanded regions in the series of expanded regions has substantially the same width.

8. The microfluidic device of any one of the preceding claims, wherein the series of expanded regions includes at least 3 expanded regions and up to 20 expanded regions.

9. The microfluidic device of any one of the preceding claims, wherein the flow channel includes more than one constriction between each expanded region in the series of expanded regions.

10. The microfluidic device of any one of the preceding claims, wherein the inlet has a width of from 2 μm to 3 μm.

11. The microfluidic device of any one of the preceding claims, wherein the microfluidic device further includes cell capture and lysis structure upstream of the inlet, the cell capture and lysis structure including:
a cell trap adjacent the flow channel inlet configured to receive and retain a cell from a fluid sample including the cell, the cell trap including:
a viewing element to permit inspection of the cell; and
an opening connected to the flow channel inlet via a passage, the opening and passage sized to impede passage of the cell therethrough;
a lysis port configured to introduce a lysis buffer to the cell trap.

12. The microfluidic device of claim 11, wherein the size of the opening is from about 10 μm to 20 μm and a width of the passage is from about 2 μm to about 3 μm.

13. The microfluidic device of any one of the preceding claims, wherein the cell trap is a rectangular prism shaped hollow formation in the microfluidic device with an open face to permit entry of a cell into the cell trap.

14. The microfluidic device of any one of the preceding claims, further including a chromosome dispensing structure downstream of the outlet, the chromosome dispensing structure including:
a dispensing channel defined between a channel inlet and a channel outlet, and having a port for receiving an individual chromosome from the outlet of the flow channel;
wherein the channel outlet is connected to a dispensing tube configured to dispense single chromosomes from the microfluidic device in the form of a fluid droplet including the single chromosome.

15. A method for separating metaphase chromosomes in a metaphase chromosome-containing fluid, the method including:
passing the metaphase chromosome-containing fluid through the microfluidic device of any one of the preceding claims at a pressure whereby the constrictions subject the metaphase chromosomes to sufficient shear stress to separate the metaphase chromosomes from one another.

16. A method for separating metaphase chromosomes in a chromosome-containing fluid, the method including:
passing a chromosome-containing fluid including metaphase chromosomes through a microfluidic device, the microfluidic device having a flow channel including:
a plurality of expanded regions located between an inlet and an outlet; and
one or more constrictions located between one or more of the expanded regions;
subjecting metaphase chromosomes, at or in the one or more constrictions, to sufficient shear stress to separate the metaphase chromosomes from one another;
dispersing the separated metaphase chromosomes in the plurality expanded regions from one another.

17. A method for separating metaphase chromosomes in a chromosome-containing fluid with a microfluidic device, the method including:
passing the fluid through a flow channel of a microfluidic device, the flow channel having a plurality of alternating constrictions and expansions;
wherein when the fluid is passed through a constriction, the method includes applying a pressure pulse to subject the metaphase chromosomes to a shear stress sufficient to separate the metaphase chromosomes from one another;
wherein when the fluid is passed through an expansion, the microfluidic device is operated at a pressure to disperse the separated chromosomes from one another.

18. The method of any one of claims 15 to 17, wherein the shear stress is from at least about 0.02 N/m² to at least about 15,000 N/m² as measured at walls of the minimum width of the constriction.

19. The method of any one of claims 15 to 18, wherein the method initially includes:
trapping a metaphase cell in a cell trap of the microfluidic device; and
introducing a lysis buffer to the metaphase cell and applying a pressure pulse to drive the metaphase cell from the cell trap and into the flow channel under sufficient shear stress to lyse the cell and provide the chromosomes in the chromosome-containing fluid.

20. The method of any one of claims 15 to 19, further including:
receiving the dispensed individual chromosomes from the outlet of the flow channel into a dispensing channel of the microfluidic device;
transporting the individual chromosomes to a dispensing tube; and
dispense single chromosomes from the microfluidic device via the dispensing tube in the form of a fluid droplet including the single chromosome.
